(19) United States
(12) Patent Application Publication
(10) Pub. No.: US 2005/0239774 A1
(43) Pub. Date: Oct. 27, 2005

(54) Biaryl diazabicycloalkane amides as nicotinic acetylcholine agonists

(76) Inventors: Glen Ernst, Wilmington, DE (US); Eifion Phillips, Wilmington, DE (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(21) Appl. No.: 10/524,484

(22) PCT Filed: Aug. 13, 2003
(86) PCT No.: PCT/SE03/01277

§ 371 Date: Feb. 14, 2005

(30) Foreign Application Priority Data
Aug. 14, 2002 (SE) 02024305

(51) Int. Cl. A61K 31/5513; A61K 31/498; C07D 487/04

(52) U.S. Cl. 514/221; 514/249; 540/567; 544/349

(57) ABSTRACT

Acetylcholine receptor ligands of formula I 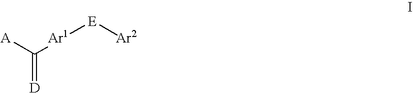
wherein A, D, Ar¹, E and Ar² are as described in the specification, diastereoisomers, enantiomers, pharmaceutically-acceptable salts, methods of making, pharmaceutical compositions containing and methods for using the same.

### TECHNICAL FIELD

This invention relates to diazabicycloalkane amides or pharmaceutically-acceptable salts thereof, processes for preparing them, pharmaceutical compositions containing them and their use in therapy. The invention also relates to compounds that are ligands for nicotinic acetylcholine receptors (nAChRs).

### BACKGROUND OF THE INVENTION

The use of compounds which bind nicotinic acetylcholine receptors in the treatment of a range of disorders involving reduced cholinergic function such as Alzheimer's disease, cognitive or attention disorders, anxiety, depression, smoking cessation, neuroprotection, schizophrenia, analgesia, Tourette's syndrome, and Parkinson's disease has been discussed in McDonald et al. (1995) “Nicotinic Acetylcholine Receptors: Molecular Biology, Chemistry and Pharmacology”, Chapter 5 in Annual Reports in Medicinal Chemistry, vol. 30, pp. 41-50, Academic Press Inc., San Diego, Calif.; and in Williams et al. (1994) “Neuronal Nicotinic Acetylcholine Receptors,” Drug News & Perspectives, vol. 7, pp. 205-223.

### DISCLOSURE OF THE INVENTION

We have invented compounds of formula I: 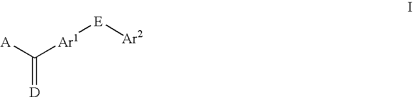
wherein:
A is a moiety of formula II: 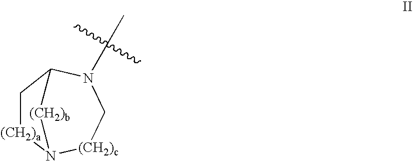
D is oxygen or sulfur;
E is a single bond, oxygen, sulfur, or NR³;
Ar¹ is a 5- or 6-membered aromatic heterocyclic ring having 1, 2 or 3 heteroatoms selected from nitrogen, oxygen or sulfur where not more than one of said heteroatoms is oxygen or sulfur, or
Ar¹ is phenyl;
Ar² is a 5- or 6-membered aromatic heterocyclic ring having 1, 2 or 3 heteroatoms selected from nitrogen, oxygen or sulfur where not more than one of said heteroatoms is oxygen or sulfur, or
Ar² is phenyl, or
Ar² is an 8- or 9-, or 10-membered fused aromatic carbocyclic ring or fused aromatic heterocyclic ring having 1, 2 or 3 heteroatoms selected from nitrogen, oxygen or sulfur where not more than one of said heteroatoms is oxygen or sulfur, or an 8- or 9-, or 10-membered aromatic carbocyclic ring;
the rings Ar¹ and Ar² are substituted with 0, 1, 2 or 3 substituents selected from: halogen, C₁₋₄alkyl, C₂₋₄alkenyl, C₂₋₄alkynyl, CN, NO₂, CF₃NR¹R², CH₂NR¹R², OR², CH₂OR² or CO₂R³;
R¹ and R² at each occurrence are independently selected from hydrogen, C₁₋₄alkyl, aryl, heteroaryl, C(O)R³, C(O)NHR³, CO₂R³ or SO₂R³, or
R¹ and R² in combination is —(CH₂)ⱼG(CH₂)ₖ— wherein G is oxygen, sulfur, NR³, or a bond;
a, b and c are each 1 or 2;
j is 2, 3 or 4;
k is 0, 1 or 2, and
R³ at each occurrence is independently selected from hydrogen, C₁₋₄alkyl, aryl, or heteroaryl;
   or a diastereoisomer, enantiomer or pharmaceutically-acceptable salt thereof.

One embodiment of the invention comprises compounds wherein D is oxygen.

Another embodiment of the invention comprises compounds wherein E is a single bond.

Yet another embodiment of the invention comprises compounds wherein E is oxygen or NR³.

A particular embodiment of the invention comprises compounds wherein A is 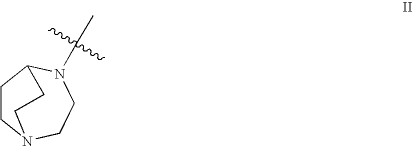
or a diastereoisomer, enantiomer or pharmaceutically-acceptable salt thereof.

Particular compounds of the invention are those wherein Ar¹ is a 5- or 6-membered aromatic heterocyclic ring having 1 or 2 heteroatoms selected from nitrogen, oxygen or sulfur where not more than one of said heteroatoms is oxygen or sulfur, or Ar¹ is phenyl.

Particular compounds of the invention are also those wherein Ar¹ is a benzene ring, furan ring or thiophene ring.

Particular compounds of the invention are also those wherein Ar² is a 5- or 6-membered aromatic heterocyclic ring having 1 or 2 heteroatoms selected from nitrogen, oxygen or sulfur where not more than one of said heteroatoms is oxygen or sulfur, or a phenyl.

Particular compounds of the invention are also those wherein Ar² is a benzene ring, furan ring, thiophene ring, or pyridine ring.

Particular compounds of the invention are also those wherein the -EAr² and the C(=D)A moieties on Ar¹ are positioned in a 1,3-relationship relative to each other.

Particular compounds of the invention are also those wherein Ar¹ or Ar² is substituted with 0 or 1 substituents selected from: halogen, C₁₋₄alkyl, C₂₋₄alkenyl, C₂₋₄alkynyl, CN, NO₂, NR¹R², CH₂NR¹R², OR³, CH₂OR³, CO₂R³, and CF₃.

Particular compounds of the invention are also those wherein A is a moiety of formula II: 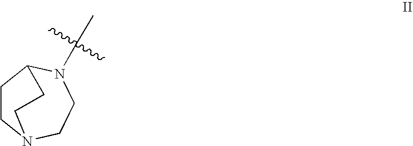
D is oxygen;
E is a single bond;
Ar¹ is a 5- or 6-membered aromatic heterocyclic ring having 1, 2 or 3 heteroatoms selected from nitrogen, oxygen or sulfur where not more than 1 of said heteroatoms is oxygen or sulfur, or
Ar¹ is phenyl
Ar² is a 5- or 6-membered aromatic heterocyclic ring having 1, 2 or 3 heteroatoms selected from nitrogen, oxygen or sulfur where not more than 1 of said heteroatoms is oxygen or sulfur, or
Ar² is phenyl,
   or a diastereoisomer, enantiomer or pharmaceutically-acceptable salt thereof.

Still more particular compounds of the invention are those wherein Ar¹ is a benzene ring, furan ring or thiophene ring.

Particular compounds of the invention are also having the groups -EAr² and —C(═O)A, positioned in a 1,3-relationship relative to each other and wherein Ar² has 0 or 1 substituents selected from: halogen, C₁₋₄alkyl, C₂₋₄alkenyl, C₂₋₄alkynyl, CN, NO₂, NR¹R², CH₂NR¹R², OR¹, CH₂OR¹, CO₂R³, and CF₃.

Most particular compounds of the invention include:
(1,4-diazabicyclo[3.2.2]non-4-yl)(biphenyl-3-yl)methanone;
(1,4-diazabicyclo[3.2.2]non-4-yl)(3-(2-pyridyl)phenyl)methanone;
(1,4-diazabicyclo[3.2.2]non-4-yl)(3-(3-pyridyl)phenyl)methanone;
(1,4-diazabicyclo[3.2.2]non-4-yl)(3-(4-pyridyl)phenyl)methanone;
(1,4-diazabicyclo[3.2.2]non-4-yl)(3-(furan-2-yl)phenyl)methanone;
(1,4-diazabicyclo[3.2.2]non-4-yl)(3-(furan-3-yl)phenyl)methanone;
(1,4-diazabicyclo[3.2.2]non-4-yl)(3-(thiophen-2-yl)phenyl)methanone;
(1,4-diazabicyclo[3.2.2]non-4-yl)(3-(thiophen-3-yl)phenyl)methanone;
(1,4-diazabicyclo[3.2.2]non-4-yl)(biphenyl-4-yl)methanone;
(1,4-diazabicyclo[3.2.2]non-4-yl)(4-(2-pyridyl)phenyl)methanone;
(1,4-diazabicyclo[3.2.2]non-4-yl)(4-(3-pyridyl)phenyl)methanone;
(1,4-diazabicyclo[3.2.2]non-4-yl)(4-(4-pyridyl)phenyl)methanone;
(1,4-diazabicyclo[3.2.2]non-4-yl)(4-(furan-2-yl)phenyl)methanone;
(1,4-diazabicyclo[3.2.2]non-4-yl)(4-(furan-3-yl)phenyl)methanone;
(1,4-diazabicyclo[3.2.2]non-4-yl)(4-(thiophen-2-yl)phenyl)methanone;
(1,4-diazabicyclo[3.2.2]non-4-yl)(4-(thiophen-3-yl)phenyl)methanone;
(1,4-diazabicyclo[3.2.2]non-4-yl)(5-phenylfuran-2-yl)methanone;
(1,4-diazabicyclo[3.2.2]non-4-yl)(5-(2-pyridyl)furan-2-yl)methanone;
(1,4-diazabicyclo[3.2.2]non-4-yl)(5-(3-pyridyl)furan-2-yl)methanone;
(1,4-diazabicyclo[3.2.2]non-4-yl)(5-(4-pyridyl)furan-2-yl)methanone;
(1,4-diazabicyclo[3.2.2]non-4-yl)(5-(furan-2-yl)furan-2-yl)methanone;
(1,4-diazabicyclo[3.2.2]non-4-yl)(5-(furan-3-yl)furan-2-yl)methanone;
(1,4-diazabicyclo[3.2.2]non-4-yl)(5-(thiophen-2-yl)furan-2-yl)methanone;
(1,4-diazabicyclo[3.2.2]non-4-yl)(5-(thiophen-3-yl)furan-2-yl)methanone;
(1,4-diazabicyclo[3.2.2]non-4-yl)(5-phenylthiophen-3-yl)methanone otherwise named (1,4-diazabicyclo[3.2.2]non-4-yl)(2-phenylthiophen-4-yl)methanone;
(1,4-diazabicyclo[3.2.2]non-4-yl)(5-(2-pyridyl)thiophen-3-yl)methanone otherwise named (1,4-diazabicyclo[3.2.2]non-4-yl)(2-(2-pyridyl)thiophenyl)methanone;
(1,4-diazabicyclo[3.2.2]non-4-yl)(5-(3-pyridyl)thiophen-3-yl)methanone otherwise named (1,4-diazabicyclo[3.2.2]non-4-yl)(2-(3-pyridyl)thiophen-4-yl)methanone;
(1,4-diazabicyclo[3.2.2]non-4-yl)(5-(4-pyridyl)thiophen-3-yl)methanone otherwise named (1,4-diazabicyclo[3.2.2]non-4-yl)(2-(4-pyridyl)thiophenyl)methanone;
(1,4-diazabicyclo[3.2.2]non-4-yl)(5-(furan-2-yl)thiophen-3-yl)methanone otherwise named (1,4-diazabicyclo[3.2.2]non-4-yl)(2-(furan-2-yl)thiophen-4-yl)methanone;
(1,4-diazabicyclo[3.2.2]non-4-yl)(5-(furan-3-yl)thiophen-3-yl)methanone otherwise named (1,4-diazabicyclo[3.2.2]non-4-yl)(2-(furan-3-yl)thiophen-4-yl)methanone;
(1,4-diazabicyclo[3.2.2]non-4-yl)(5-(thiophen-2-yl)thiophen-3-yl)methanone otherwise named (1,4-diazabicyclo[3.2.2]non-4-yl)(2-(thiophen-2-yl)thiophen-4-yl)methanone;
(1,4-diazabicyclo[3.2.2]non-4-yl)(5-(thiophen-3-yl)thiophen-3-yl)methanone otherwise named (1,4-diazabicyclo[3.2.2]non-4-yl)(2-(thiophen-3-yl)thiophen-4-yl)methanone;
(1,4-diazabicyclo[3.2.2]non-4-yl)(4-phenylfuran-2-yl)methanone;
(1,4-diazabicyclo[3.2.2]non-4-yl)(4-(2-pyridyl)furan-2-yl)methanone;
(1,4-diazabicyclo[3.2.2]non-4-yl)(4-(3-pyridyl)furan-2-yl)methanone;
(1,4-diazabicyclo[3.2.2]non-4-yl)(4-(4-pyridyl)furan-2-yl)methanone;
(1,4-diazabicyclo[3.2.2]non-4-yl)(4-(furan-2-yl)furan-2-yl)methanone;
(1,4-diazabicyclo[3.2.2]non-4-yl)(4-(furan-3-yl)furan-2-yl)methanone;
(1,4-diazabicyclo[3.2.2]non-4-yl)(4-(thiophen-2-yl)furan-2-yl)methanone, and
(1,4-diazabicyclo[3.2.2]non-4-yl)(4-(thiophen-3-yl)furan-2-yl)methanone.

Other compounds of the invention are:
(1,4-diazabicyclo[3.2.2]non-4-yl)(5-phenylthiophen-2-yl)methanone;
(1,4-diazabicyclo[3.2.2]non-4-yl)(5-(2-pyridyl)thiophen-2-yl)methanone;
(1,4-diazabicyclo[3.2.2]non-4-yl)(5-(3-pyridyl)thiophen-2-yl)methanone;
(1,4-diazabicyclo[3.2.2]non-4-yl)(5-(4-pyridyl)thiophen-2-yl)methanone;
(1,4-diazabicyclo[3.2.2]non-4-yl)(5-(furan-2-yl)thiophen-2-yl)methanone;
(1,4-diazabicyclo[3.2.2]non-4-yl)(5-(furan-3-yl)thiophen-2-yl)methanone;
(1,4-diazabicyclo[3.2.2]non-4-yl)(5-(thiophen-2-yl)thiophen-2-yl)methanone, and
(1,4-diazabicyclo[3.2.2]non-4-yl)(5-(thiophen-3-yl)thiophen-2-yl)methanone.

Yet other compounds of the invention are:
(1,4-diazabicyclo[3.2.2]non-4-yl)(2-phenylfuran-4-yl)methanone;
(1,4-diazabicyclo[3.2.2]non-4-yl)(2-(2-pyridyl)furan-4-yl)methanone;
(1,4-diazabicyclo[3.2.2]non-4-yl)(2-(3-pyridyl)furan-4-yl)methanone;
(1,4-diazabicyclo[3.2.2]non-4-yl)(2-(4-pyridyl)furan-4-yl)methanone;
(1,4-diazabicyclo[3.2.2]non-4-yl)(2-(furan-2-yl)furan-4-yl)methanone;
(1,4-diazabicyclo[3.2.2]non-4-yl)(2-(furan-3-yl)furan-4-yl)methanone;
(1,4-diazabicyclo[3.2.2]non-4-yl)(2-(thiophen-2-yl)furan-4-yl)methanone;
(1,4-diazabicyclo[3.2.2]non-4-yl)(2-(thiophen-3-yl)furan-4-yl)methanone;
(1,4-diazabicyclo[3.2.2]non-4-yl)(4-phenylthiophen-2-yl)methanone;
(1,4-diazabicyclo[3.2.2]non-4-yl)(4-(2-pyridyl)thiophen-2-yl)methanone;
(1,4-diazabicyclo[3.2.2]non-4-yl)(4-(3-pyridyl)thiophen-2-yl)methanone;
(1,4-diazabicyclo[3.2.2]non-4-yl)(4-(4-pyridyl)thiophen-2-yl)methanone;
(1,4-diazabicyclo[3.2.2]non-4-yl)(4-(furan-2-yl)thiophen-2-yl)methanone;
(1,4-diazabicyclo[3.2.2]non-4-yl)(4-(furan-3-yl)thiophen-2-yl)methanone;
(1,4-diazabicyclo[3.2.2]non-4-yl)(4-(thiophen-2-yl)thiophen-2-yl)methanone, and
(1,4-diazabicyclo[3.2.2]non-4-yl)(4-(thiophen-3-yl)thiophen-2-yl)methanone.

All embodiments and particular forms of the invention encompass all enantiomers, diastereoisomers and pharmaceutically-acceptable derivatives and salts of compounds thereof.

Compounds of the invention are potent ligands for nicotinic acetylcholine receptors.

Pharmaceutically-acceptable derivatives include solvates and salts. For example, the compounds of formula I can form acid addition salts with acids, such as the conventional pharmaceutically-acceptable acids, for example, maleic, hydrochloric, hydrobromic, phosphoric, acetic, fumaric, salicylic, citric, lactic, mandelic, tartaric and methanesulfonic acids.

Compounds of the invention are useful in the treatment or prophylaxis of human diseases or conditions in which activation of the α7 nicotinic receptor is beneficial as well as in the treatment or prophylaxis of psychotic disorders or intellectual impairment disorders. Examples of such conditions, diseases or disorders are Alzheimers disease, learning deficit, cognition deficit, attention deficit, memory loss, Attention Deficit Hyperactivity Disorder, Anxiety, schizophrenia, mania or manic depression, Parkinson's disease, Huntington's disease, Tourette's syndrome, neurodegenerative disorders in which there is loss of cholinergic synapse, jetlag, cessation of smoking, nicotinic addiction including that resulting from exposure to products containing nicotine, pain, for ulcerative colitis and irritable bowel disease.

As used herein, unless otherwise indicated, “C₁₋₄alkyl” includes but is not limited to methyl, ethyl, n-propyl, n-butyl, i-propyl, i-butyl, t-butyl, s-butyl moieties, whether alone or part of another group, C₁₋₄alkyl groups may be straight-chained or branched, and C₃₋₄ alkyl groups include the cyclic alkyl moieties cyclopropyl and cyclobutyl. Alkyl groups referred to herein may have 1, 2 or 3 halogen substituents.

As used herein, unless otherwise indicated, “C₂₋₄alkenyl” includes but is not limited to 1-propenyl, 2-propenyl, 1-butenyl, 2-butenyl and 3-butenyl.

As used herein, unless otherwise indicated, “C₂₋₄alkynyl” includes but is not limited to ethynyl, 1-propynyl, 2-propynyl, 1-butynyl, 2-butynyl and 3-butynyl.

As used herein, unless otherwise indicated, aryl refers to a phenyl ring which may have 1, 2 or 3 substituents selected from: halogen, C₁₋₄alkyl, C₂₋₄alkenyl, C₂₋₄alkynyl, C₁₋₄aklyl, CN, NO₂, and CF₃.

As used herein, unless otherwise indicated, heteroaryl refers to a 5- or 6-membered aromatic or heteroaromatic ring having 1, 2 or 3 heteroatoms selected from nitrogen oxygen and sulfur, provided that heteroaromatic rings contains at least one nitrogen, oxygen, or sulfur atom.

As used herein, unless otherwise indicated, halogen refers to fluorine, chlorine, bromine, or iodine.

Methods of Preparation

In the reaction schemes and text that follow, A, E, Ar¹, and Ar² unless otherwise indicated, are as defined above for formula I.

The compounds of formula I in which E represent a single bond may be prepared according to the methods outlined in Scheme 1.

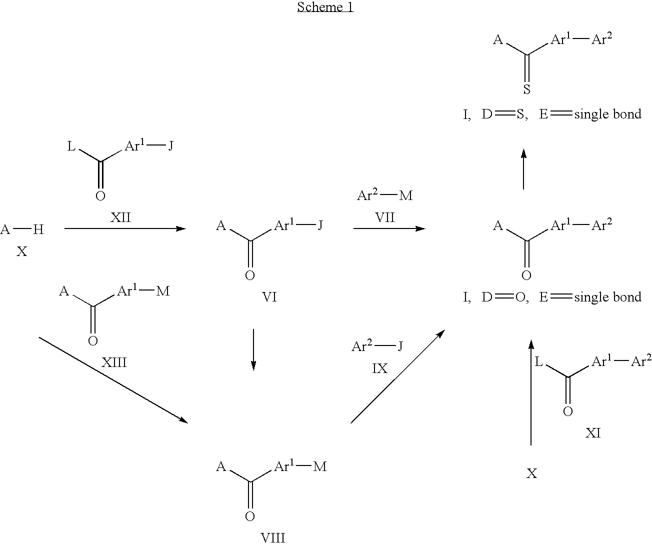

Compounds of formula I wherein D is oxygen and E is a single bond may be prepared from compounds of formula VI wherein J is a halogen or an OSO₂CF₃ substituent at the position of ring Ar¹ at which the bond to ring Ar² is formed, by reaction with an appropriate organometallic compound of formula VII in the presence of a suitable organometallic catalyst and solvent. Suitable compounds of formula VII include boronic acids, in which M is B(OH)₂ and organotin compounds, in which M is a suitable trialkylstannyl group, for example trimethylstannyl or tri-n-butylstannyl. Suitable organometallic catalysts include palladium (0) complexes, for example tetrakis(triphenylphosphine)palladium(0) or a combination of tris(dibenzylideneacetone)dipalladium(0) and a suitable triarylphosphine or triarylarsine ligand, for example triphenylphosphine, tri(o-tolyl)phosphine or triphenylarsine.

Suitable solvents include inert ether solvents, for example 1,2-dimethoxyethane, tetrahydrofuran, or 1,4-dioxane, or alcohols, such as ethanol, or mixtures thereof. If the compound of formula VII is a boronic acid, the presence of a suitable base in addition to the other reagents is preferred. Suitable bases include sodium carbonate, cesium carbonate, and barium hydroxide. The reaction is carried out at a temperature of 0-120° C., and preferably at a temperature of 60-120° C.

Compounds of formula I wherein D is oxygen and E is a single bond may also be prepared from organometallic compounds of formula VIII by reaction with a compound of formula IX in which J is a halogen or OSO₂CF₃ in the presence of a suitable organometallic catalyst and solvent. Suitable compounds of formula VIII include boronic acids, in which M is B(OH)₂ and organotin compounds, in which M is a suitable trialkylstannyl group, for example trimethylstannyl or tri-n-butylstannyl.

Suitable organometallic catalysts include palladium (0) complexes, for example tetrakis(triphenylphosphine)palladium (0) or a combination of tris(dibenzylideneacetone)dipalladium (0) and a suitable triarylphosphine or triarylarsine ligand, for example triphenylphosphine, tri(o-tolyl)phosphine or triphenylarsine.

Suitable solvents include inert ether solvents, for example 1,2-dimethoxyethane, tetrahydrofuran, or 1,4-dioxane, or alcohols, such as ethanol, or mixtures thereof.

If the compound of formula VIII is a boronic acid, the presence of a suitable base in addition to the other reagents is preferred. Suitable bases include sodium carbonate, cesium carbonate, and barium hydroxide. The reaction is carried out at a temperature of 0-120° C., and preferably at a temperature of 60-120° C.

Compounds of formula I wherein D is oxygen and E is a single bond may also be prepared from compounds of formula X by reaction with a suitable compound of formula XI, wherein L is a suitable leaving group, using a suitable acylation procedure. Suitable leaving groups L include: OH, halogen, Oalkyl, Oaryl, OCOalkyl, OCOaryl. A suitable acylation procedure involves treatment of a compound of formula X with a compound of formula XI at 0-120° C. in a suitable solvent. The presence of a base, or, when Y=OH, a coupling agent, may also be necessary for the reaction to occur. Suitable bases for the reaction include: 4-(N,N-dimethylamino)pyridine, pyridine, triethylamine, N,N-diisopropylethylamine. The preferred base is N,N-diisopropylethylamine. Suitable coupling agents when L=OH include: carbodiimides, for example 1,3-dicyclohexylcarbodiimide or 1-(3-dimethylaminopropyl-3-ethylcarbodiimide hydrochloride; phosphonium reagents, for example benzotriazol-1-yloxytris(dimethylamino)phosphonium hexafluorophosphate or benzotriazol-1-yloxytripyrrolidinophosphonium hexafluorophosphate; and uronium reagents, for example O-benzotriazol-1-yl-N,N,N′,N′-tetramethyluronium tetrafluoroborate. The preferred coupling agent is O-benzotriazol-1-yl-N,N,N′,N′-tetramethyluronium tetrafluoroborate. Suitable solvents for the reaction include N,N-dimethylformamide, dimethylsulfoxide, tetrahydrofuran, or chloroform. The preferred solvent is N,N-dimethylformamide. The reaction is preferably performed at a temperature of 0-50° C., and most preferably at a temperature of 20-30° C.

Compounds of formula I in which D is sulfur and E is a single bond may be prepared from compounds of formula I in which D is oxygen and E is a single bond by reaction with a suitable sulfide in a suitable solvent. The preferred sulfides are phosphorus sulfides, in particular 4-methoxyphenylthionophosphine sulfide dimer (“Lawesson's Reagent”), and diphosphorus pentasulfide. Suitable solvents for the reaction include aryl hydrocarbon solvents, for example toluene or xylene. The reaction is performed at a temperature of 0-200° C., and preferably at a temperature of 50-180° C.

Certain compounds of formula VI wherein J is halogen may be prepared from compounds of formula VI wherein J is hydrogen by reaction with a suitable halogenating agent in a suitable solvent. Suitable halogenating agents include bromine. Suitable solvents include acetic acid. The reaction is preferably performed at a temperature of 0-50° C., and most preferably at a temperature of 0-25° C.

Compounds of formula VI wherein J is OSO₂CF₃ may be prepared from compounds of formula VI wherein J is OH by reaction with trifluoromethanesulfonic anhydride or other trifluoromethanesulfonylating agent in the presence of a base and a suitable solvent. Suitable bases include pyridine, and 2,6-di-t-butylpyridine. The reaction is preferably performed at a temperature of −78 to 120° C., and most preferably at a temperature of −78 to 0° C.

Compounds of formula VI wherein J is hydrogen, halogen, OH, or OSO₂CF₃ may be prepared from compounds of formula X by reaction with a suitable compound of formula XII, wherein L is a suitable leaving group and J is hydrogen, halogen, OH, or OSO₂CF₃, using a suitable acylation procedure. Suitable leaving groups L include: OH, halogen, Oalkyl, Oaryl, OCOalkyl, OCOaryl. A suitable acylation procedure involves treatment of a compound of formula X with a compound of formula XII at 0-120° C. in a suitable solvent. The presence of a base, or, when L=OH, a coupling agent, may also be necessary for the reaction to occur. Suitable bases for the reaction include: 4-(N,N-dimethylamino)pyridine, pyridine, triethylamine, N,N-diisopropylethylamine. The preferred base is N,N-diisopropylethylamine. Suitable coupling agents when Y=OH include: carbodiimides, for example 1,3-dicyclohexylcarbodiimide or 1-(3-dimethylaminopropyl-3-ethylcarbodiimide hydrochloride; phosphonium reagents, for example benzotriazol-1-yloxytris(dimethylamino)phosphonium hexafluorophosphate or benzotriazol-1-yloxytripyrrolidinophosphonium hexafluorophosphate; and uronium reagents, for example O-benzotriazol-1-yl-N,N,N′,N′-tetramethyluronium tetrafluoroborate. The preferred coupling agent is O-benzotriazol-1-yl-N,N,N′,N′-tetramethyluronium tetrafluoroborate. Suitable solvents for the reaction include N,N-dimethylformamide, dimethylsulfoxide, tetrahydrofuran, or chloroform. The preferred solvent is N,N-dimethylformamide. The reaction is preferably performed at a temperature of 0-50° C., and most preferably at a temperature of 20-30° C.

Compounds of formula VIII in which M is B(OH)₂ may be prepared from compounds of formula VI in which J is hydrogen, halogen, or OSO₂CF₃ by methods known to one skilled in the art. For example compounds of formula VI in which J is hydrogen or halogen may be converted to compounds of formula VIII in which M is B(OH)₂ via conversion to the corresponding aryllithium or arylmagnesium compounds followed by reaction with trimethylborate and subsequent hydrolysis of the resulting borate ester. The reaction is performed in a suitable inert solvent, for example, tetrahydrofuran. Alternatively, compounds of formula VI wherein J is halogen or OSO₂CF₃ may be converted to compounds of formula VIII in which M is B(OH)₂ via reaction with bis(pinacolato)diboron and an organometallic catalyst, followed by hydrolysis of the resulting borate ester. For typical procedures for effecting such conversions, see, for example, Organic Syntheses, 1963, Coll. Vol. 4, 68; J. Org. Chem. 1995, 60, 7508.

Compounds of formula VIII in which M is a trialkylstannyl group may be prepared from compounds of formula VI in which J is hydrogen, halogen, or OSO₂CF₃ by methods known to one skilled in the art. For example compounds of formula VI in which J is hydrogen or halogen may be converted to compounds of formula VIII in which M is a trialkylstannyl group via conversion to the corresponding aryllithium or arylmagnesium compounds followed by reaction with an appropriate trialkylstannyl halide. The reaction is performed in a suitable inert solvent, for example, tetrahydrofuran. The reaction is performed at a temperature of −78° C. to 20° C., preferably at −78° C. to 0° C. Alternatively, compounds of formula VI wherein J is halogen or OSO₂CF₃ may be converted to compounds of formula VIII in which M is a trialkylstannyl group via reaction with the appropriate bis(trialkyltin). The reaction is performed in a suitable inert solvent, for example tetrahydrofuran, in the presence of a suitable organometallic catalyst, for example tetrakis(triphenylphosphine). The reaction is performed at a temperature of 0° C. to 150° C., preferably at 20° C. to 100° C.

Compounds of formula VIII wherein M is B(OH)₂ or a trialkylstannyl group may be prepared from compounds of formula X by reaction with a suitable compound of formula XIII, wherein L is a suitable leaving group M is B(OH)₂ or a trialkylstannyl group, using a suitable acylation procedure. Suitable leaving groups L include: OH, halogen, Oalkyl, Oaryl, OCOalkyl, OCOaryl. A suitable acylation procedure involves treatment of a compound of formula X with a compound of formula XIII at 0-120° C. in a suitable solvent. The presence of a base, or, when L=OH, a coupling agent, may also be necessary for the reaction to occur. Suitable bases for the reaction include: 4-(N,N-dimethylamino)pyridine, pyridine, triethylamine, N,N-diisopropylethylamine. The preferred base is N,N-diisopropylethylamine. Suitable coupling agents when L=OH include: carbodiimides, for example 1,3-dicyclohexylcarbodiimide or 1-(3-dimethylaminopropyl-3-ethylcarbodiimide hydrochloride; phosphonium reagents, for example benzotriazol-1-yloxytris(dimethylamino)phosphoniurn hexafluorophosphate or benzotriazol-1-yloxytripyrrolidinophosphonium hexafluorophosphate; and uronium reagents, for example O-benzotriazol-1-yl-N,N,N′,N′-tetramethyluronium tetrafluoroborate. The preferred coupling agent is O-benzotriazol-1-yl-N,N,N′,N′-tetramethyluronium tetrafluoroborate. Suitable solvents for the reaction include N,N-dimethylformamide, dimethylsulfoxide, tetrahydrofuran, or chloroform. The preferred solvent is N,N-dimethylformamide. The reaction is preferably performed at a temperature of 0-50° C., and most preferably at a temperature of 20-30° C.

Compounds of formula XI may be prepared from compounds of formula XII wherein J is a halogen or OSO₂CF₃ substituent at the position of ring Ar¹ at which the bond to ring Ar² is formed, by reaction with an appropriate organometallic compound of formula VII in the presence of a suitable organometallic catalyst and solvent. Suitable compounds of formula VII include boronic acids, in which M is B(OH)₂ and organotin compounds, in which M is a suitable trialkylstannyl group, for example trimethylstannyl or tri-n-butylstannyl. Suitable organometallic catalysts include palladium (0) complexes, for example tetrakis(triphenylphosphine)palladium (0) or a combination of tris(dibenzylidieneacetone)dipalladium (0) and a suitable triarylphosphine or triarylarsine ligand, for example triphenylphosphine, tri(o-tolyl)phosphine or triphenylarsine. Suitable solvents include inert ether solvents, for example 1,2-dimethoxyethane, tetrahydrofuran, or 1,4-dioxane, or alcohols, such as ethanol, or mixtures thereof. If the compound of formula VII is a boronic acid, the presence of a suitable base in addition to the other reagents is preferred. Suitable bases include sodium carbonate, cesium carbonate, and barium hydroxide. The reaction is carried out at a temperature of 0-120° C., and preferably at a temperature of 60-120° C.

Compounds of formula XI may also be prepared from organometallic compounds of formula XIII by reaction with a compound of formula IX in which J is a halogen or OSO₂CF₃ in the presence of a suitable organometallic catalyst and solvent. Suitable compounds of formula XIII include boronic acids, in which M is B(OH)₂ and organotin compounds, in which M is a suitable trialkylstannyl group, for example trimethylstannyl or tri-n-butylstannyl. Suitable organometallic catalysts include palladium (0) complexes, for example tetrakis(triphenylphosphine)palladium (0) or a combination of tris(dibenzylideneacetone)dipalladium (0) and a suitable triarylphosphine or triarylarsine ligand, for example triphenylphosphine, tri(o-tolyl)phosphine or triphenylarsine. Suitable solvents include inert ether solvents, for example 1,2-dimethoxyethane, tetrahydrofuran, or 1,4-dioxane, or alcohols, such as ethanol, or mixtures thereof. If the compound of formula VIII is a boronic acid, the presence of a suitable base in addition to the other reagents is preferred. Suitable bases include sodium carbonate, cesium carbonate, and barium hydroxide. The reaction is carried out at a temperature of 0-120° C., and preferably at a temperature of 60-120° C.

Compounds of formula VII and compounds of formula XIII are either commercially available, or may be prepared by methods known to one skilled in the art. In particular, methods are known to one skilled in the art for the conversion of aryl halides or heteroaryl halides to aryl or heteroaryl boronic acids or aryl or heteroaryl trialkylstannanes, providing methods for the conversion of compounds of formula IX in which J is halogen to compounds of formula VII and compounds of formula XII in which J is halogen to compounds of formula XIII. For example, boronic acids may be synthesized from aryl or heteroaryl halides via conversion to the aryllithium or arylmagnesium compounds followed by reaction with trimethylborate, or via reaction with bis(pinacolato)diboron and an organometallic catalyst, followed by hydrolysis of the resulting borate ester (see, for example, Organic Syntheses, 1963, Coll. Vol. 4, 68; J. Org. Chem. 1995, 60, 7508). Trialkylstannanes may be synthesized from aryl or heteroaryl halides via conversion to the aryllithium or arylmagnesium compounds followed by reaction with the appropriate chlorotrialkyltin, or via reaction with the appropriate bis(trialkyltin) and an organometallic catalyst.

The compounds of formula I in which E is oxygen, sulfur, or NR³ may be prepared according to the methods outlined in Scheme 2.

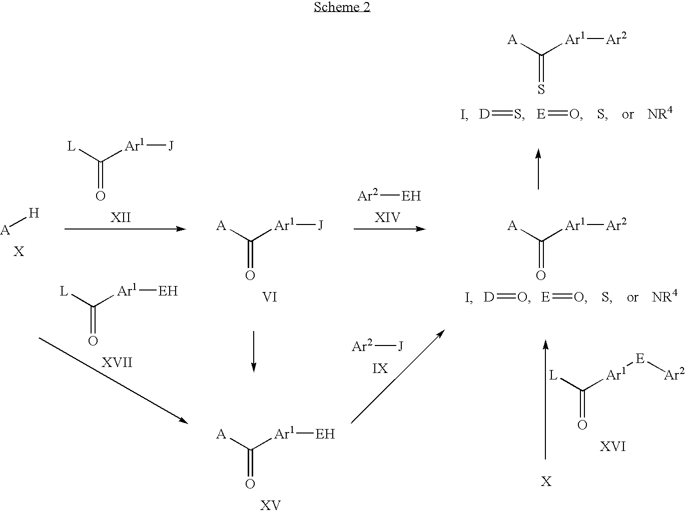

Compounds of formula I wherein D is oxygen and E is NR³ may be prepared from compounds of formula VI wherein J is a halogen or OSO₂CF₃ substituent at the position of ring Ar¹ at which the bond to nitrogen is formed, by reaction with an appropriate amine of formula XIV in which EH is NHR³. The reaction may be performed by heating in an inert solvent in the presence of a suitable strong base. Suitable inert solvents include ether solvents, for example tetrahydrofuran, 1,4-dioxane, 1,2-dimethoxyethane, or di(2-methoxyethyl)ether, a hydrocarbon solvent, for example benzene or toluene, or an amide solvent, for example dimethylformamide, or N-methyl-2-pyrrolidinone. The preferred solvent is tetrahydrofuran. Suitable strong bases include alkali metal alkoxide or amide bases, for example sodium t-butoxide or potassium t-butoxide, lithium bis(trimethylsilyl)amide, or lithium diisopropylamide. The preferred strong base is sodium t-butoxide. The reaction may require, and is preferably performed in, the presence of an organometallic catalyst. Suitable organometallic catalysts include complexes of palladium (0) with a suitable phosphine ligand, preferably a triarylphosphine ligand, and most preferably a bidentate triarylphosphine ligand. Preferred ligands include 2,2′-bis(diphenylphosphino)-1,1′-binaphthyl or 1,1′-bis(diphenylphosphino)ferrocene. The catalyst may be synthesized by the combination of a suitable source of palladium (0), for example tris(dibenzylideneacetone)dipalladium (0), with the phosphine ligand, and may either be pre-formed or formed in situ by including the palladium source and phophine ligand in the reaction mixture. The reaction is carried out at a temperature of 0-150° C., and preferably at a temperature of 60-120° C.

Compounds of formula I wherein D is oxygen and E is R³ may also be prepared from compounds of formula IX wherein J is a halogen or OSO₂CF₃ substituent at the position of ring Ar² at which the bond to nitrogen is formed, by reaction with an appropriate amine of formula XV in which EH is NHR³. The reaction may be performed by heating in an inert solvent in the presence of a suitable strong base. Suitable inert solvents include ether solvents, for example tetrahydrofuran, 1,4-dioxane, 1,2-dimethoxyethane, or di(2-methoxyethyl)ether, a hydrocarbon solvent, for example benzene or toluene, or an amide solvent, for example dimethylformamide, or N-methyl-2-pyrrolidinone. The preferred solvent is tetrahydrofuran. Suitable strong bases include alkali metal alkoxide or amide bases, for example sodium t-butoxide or potassium t-butoxide, lithium bis(trimethylsilyl)amide, or lithium diisopropylamide. The preferred strong base is sodium t-butoxide. The reaction may require, and is preferably performed in, the presence of an organometallic catalyst. Suitable organometallic catalysts include complexes of palladium (0) with a suitable phosphine ligand, preferably a triarylphosphine ligand, and most preferably a bidentate triarylphosphine ligand. Preferred ligands include 2,2′-bis(diphenylphosphino)-1,1′-binaphthyl or 1,1′-bis(diphenylphosphino)ferrocene. The catalyst may be synthesized by the combination of a suitable source of palladium (0), for example tris(dibenzylideneacetone)dipalladium (0), with the phosphine ligand, and may either be pre-formed or formed in situ by including the palladium source and phophine ligand in the reaction mixture. The reaction is carried out at a temperature of 0-150° C., and preferably at a temperature of 60-120° C.

Compounds of formula I wherein D is oxygen and E is oxygen or sulfur may be prepared from compounds of formula VI wherein J is a halogen or OSO₂CF₃ substituent at the position of ring Ar¹ at which the bond to oxygen is formed, by reaction with an appropriate compound of formula XIV in which EH is OH or SH. The reaction may be performed by heating in an inert solvent in the presence of a suitable base. The reaction may require, and is preferably performed in, the presence of a catalyst. Suitable inert solvents include ether solvents, for example tetrahydrofuran, 1,4-dioxane, 1,2-dimethoxyethane, or di(2-methoxyethyl)ether, an amide solvent, for example dimethylformamide, or N-methyl-2-pyrrolidinone, or a basic heterocyclic aromatic solvent, for example pyridine. The preferred solvent is pyridine. Suitable bases include alkali metal alkoxides, or alkali metal carbonates, for example potassium carbonate. Suitable organometallic catalysts include copper or its salts, preferably copper (I) salts, and most preferably copper (I) iodide. The reaction is carried out at a temperature of 0-150° C., and preferably at a temperature of 100-150° C.

Compounds of formula I wherein D is oxygen and E is oxygen or sulfur may also be prepared from compounds of formula IX wherein J is a halogen or OSO₂CF₃ substituent at the position of ring Ar² at which the bond to nitrogen is formed, by reaction with an appropriate compound of formula XV in which EH is OH or SH. The reaction may be performed by heating in an inert solvent in the presence of a suitable base. The reaction may require, and is preferably performed in, the presence of a catalyst. Suitable inert solvents include ether solvents, for example tetrahydrofuran, 1,4-dioxane, 1,2-dimethoxyethane, or di(2-methoxyethyl)ether, an amide solvent, for example N,N-dimethylformamide, or N-methylpyrrolidinone, or a basic heterocyclic aromatic solvent, for example pyridine. The preferred solvent is pyridine. Suitable bases include alkali metal alkoxides, or alkali metal carbonates, for example potassium carbonate. Suitable organometallic catalysts include copper or its salts, preferably copper (I) salts, and most preferably copper (I) iodide. The reaction is carried out at a temperature of 0-150° C., and preferably at a temperature of 100-150° C.

Compounds of formula I wherein D is oxygen and E is oxygen, sulfur, or NR³ may also be prepared from compounds of formula X by reaction with a suitable compound of formula XVI, wherein E is oxygen, sulfur, or NR³ and L is a suitable leaving group, using a suitable acylation procedure. Suitable leaving groups L include: OH, halogen, Oalkyl, Oaryl, OCOalkyl, OCOaryl. A suitable acylation procedure involves treatment of a compound of formula X with a compound of formula XI at 0-120° C. in a suitable solvent. The presence of a base, or, when Y=OH, a coupling agent, may also be necessary for the reaction to occur. Suitable bases for the reaction include: 4-(N,N-dimethylamino)pyridine, pyridine, triethylamine, N,N-diisopropylethylamine. The preferred base is N,N-diisopropylethylamine. Suitable coupling agents when L=OH include: carbodiimides, for example 1,3-dicyclohexylcarbodiimide or 1-(3-dimethylaminopropyl-3-ethylcarbodiimide hydrochloride; phosphoniurn reagents, for example benzotriazol-1-yloxytris(dimethylamino)phosphonium hexafluorophosphate or benzotriazol-1-yloxytripyrrolidinophosphonium hexafluorophosphate; and uronium reagents, for example O-benzotriazol-1-yl-N,N,N′,N′-tetramethyluronium tetrafluoroborate. The preferred coupling agent is O-benzotriazol-1-yl-N,N,N′,N′-tetramethyluronium tetrafluoroborate. Suitable solvents for the reaction include N,N-dimethylformamide, dimethylsulfoxide, tetrahydrofuran, or chloroform. The preferred solvent is N,N-dimethylformamide. The reaction is preferably performed at a temperature of 0-50° C., and most preferably at a temperature of 20-30° C.

Compounds of formula XV wherein EH is OH, SH, or NHR³ may be prepared from compounds of formula X by reaction with a suitable compound of formula XVII, wherein L is a suitable leaving group and EH is OH, SH or NHR³, using a suitable acylation procedure. Suitable leaving groups L include: OH, halogen, Oalkyl, Oaryl, OCOalkyl, OCOaryl. A suitable acylation procedure involves treatment of a compound of formula X with a compound of formula XVII at 0-120° C. in a suitable solvent. The presence of a base, or, when L=OH, a coupling agent, may also be necessary for the reaction to occur. Suitable bases for the reaction include: 4-(N,N-dimethylamino)pyridine, pyridine, triethylamine, N,N-diisopropylethylamine. The preferred base is N,N-diisopropylethylamine. Suitable coupling agents when L=OH include: carbodiimides, for example 1,3-dicyclohexylcarbodiimide or 1-(3-dimethylaminopropyl-3-ethylcarbodiimide hydrochloride; phosphonium reagents, for example benzotriazol-1-yloxytris(dimethylamino)phosphonium hexafluorophosphate or benzotriazol-1-yloxytripyrrolidinophosphonium hexafluorophosphate; and uronium reagents, for example O-benzotriazol-1-yl-N,N,N′,N′-tetramethyluronium tetrafluoroborate. The preferred coupling agent is O-benzotriazol-1-yl-N,N,N′,N′-tetramethyluronium tetrafluoroborate. Suitable solvents for the reaction include N,N-dimethylformamide, dimethylsulfoxide, tetrahydrofuran, or chloroform. The preferred solvent is N,N-dimethylformamide. The reaction is preferably performed at a temperature of 0-50° C., and most preferably at a temperature of 20-30° C.

Compounds of formula I, XIV, XV or XVII in which E is NR³ and R³ is an alkyl group may be prepared from compounds of the corresponding formula wherein R³ is hydrogen by a suitable alkylation procedure. Typical alkylation procedures include treatment with an appropriate alkyl halide or sulfonate ester and base, for example sodium hydride, in a suitable solvent, for example N,N-dimethylformamide, or reductive alkylation using the appropriate aldehyde or ketone together with a suitable reducing agent in the presence of an acidic catalyst and in an inert solvent. The preferred method is reductive alkylation. Suitable reducing agents include sodium borohydride and sodium cyanoborohydride. The preferred reducing agent is sodium borohydride. Suitable inert solvents include water, methanol or ethanol. The preferred solvent is methanol. Suitable acidic catalysts include acetic acid or zinc chloride. The preferred acidic catalyst is acetic acid. The reaction is usually conducted at a temperature of 0-100° C., and preferably at 20-65° C.

Compounds of formula I, XIV, XV or XVII in which E is NR³ and R³ is an aryl or heteroaryl group may be prepared from compounds of the corresponding formula wherein R³ is hydrogen by reaction with an appropriate aromatic or heteroaromatic halide or trifluoromethanesulfonate. The reaction may be performed by heating in an inert solvent in the presence of a suitable strong base. Suitable inert solvents include ether solvents, for example tetrahydrofuran, 1,4-dioxane, 1,2-dimethoxyethane, or di(2-methoxyethyl)ether, a hydrocarbon solvent, for example benzene or toluene, or an amide solvent, for example N,N-dimethylformamide, or N-methyl-2-pyrrolidinone. The preferred solvent is tetrahydrofuran. Suitable strong bases include alkali metal alkoxide or amide bases, for example sodium t-butoxide or potassium t-butoxide, lithium bis(trimethylsilyl)amide, or lithium diisopropylamide. The preferred strong base is sodium t-butoxide. The reaction may require, and is preferably performed in, the presence of an organometallic catalyst. Suitable organometallic catalysts include complexes of palladium (0) with a suitable phosphine ligand, preferably a triarylphosphine ligand, and most preferably a bidentate triarylphosphine ligand. Preferred ligands include 2,2′-bis(diphenylphosphino)-1,1′-binaphthyl or 1,1′-bis(diphenylphosphino)ferrocene. The catalyst may be synthesized by the combination of a suitable source of palladium (0), for example tris(dibenzylidieneacetone)dipalladium (0), with the phosphine ligand, and may either be preformed or formed in situ by including the palladium source and phophine ligand in the reaction mixture. The reaction is carried out at a temperature of 0-150° C., and preferably at a temperature of 60-120° C.

Compounds of formula I in which D is sulfur and E is oxygen or NR¹ may be prepared from compounds of formula I in which D is oxygen and E is oxygen, or NR³ by reaction with a suitable sulfide in a suitable solvent. The preferred sulfides are phosphorus sulfides, in particular 4-methoxyphenylthionophosphine sulfide dimer (“Lawesson's Reagent”), and diphosphorus pentasulfide. Suitable solvents for the reaction include aryl hydrocarbon solvents, for example toluene or xylene. The reaction is performed at a temperature of 0-200° C., and preferably at a temperature of 50-180° C.

Compounds of formula XVI wherein D is oxygen and E is NR³ may be prepared from compounds of formula XII wherein J is a halogen or OSO₂CF₃ substituent at the position of ring Ar¹ at which the bond to nitrogen is formed, by reaction with an appropriate amine of formula XIV in which EH is NHR³, or, alternatively, from compounds of formula XVII in which EH is NHR³ by reaction with an appropriate compound of formula IX wherein J is a halogen or OSO₂CF₃ substituent at the position of ring Ar² at which the bond to nitrogen is formed. The reaction may be performed by heating in an inert solvent in the presence of a suitable strong base. Suitable inert solvents include ether solvents, for example tetrahydrofuran, 1,4-dioxane, 1,2-dimethoxyethane, or di(2-methoxyethyl)ether, a hydrocarbon solvent, for example benzene or toluene, or an amide solvent, for example N,N-dimethylformamide, or N-methyl-2-pyrrolidinone. The preferred solvent is tetrahydrofuran. Suitable strong bases include alkali metal alkoxide or amide bases, for example sodium t-butoxide or potassium t-butoxide, lithium bis(trimethylsilyl)amide, or lithium diisopropylamide. The preferred strong base is sodium t-butoxide. The reaction may require, and is preferably performed in, the presence of an organometallic catalyst. Suitable organometallic catalysts include complexes of palladium (0) with a suitable phosphine ligand, preferably a triarylphosphine ligand, and most preferably a bidentate triarylphosphine ligand. Preferred ligands include 2,2′-bis(diphenylphosphino)-1,1′-binaphthyl or 1,1′-bis(diphenylphosphino)ferrocene. The catalyst may be synthesized by the combination of a suitable source of palladium (0), for example tris(dibenzylideneacetone)dipalladium (0), with the phosphine ligand, and may either be pre-formed or formed in situ by including the palladium source and phophine ligand in the reaction mixture. The reaction is carried out at a temperature of 0-150° C., and preferably at a temperature of 60-120° C.

Compounds of formula XVI wherein D is oxygen and E is oxygen or sulfur may be prepared from compounds of formula XII wherein J is a halogen or OSO₂CF₃ substituent at the position of ring Ar¹ at which the bond to oxygen or sulfur is formed, by reaction with an appropriate compound of formula X[V in which EH is OH or SH, or, alternatively, from compounds of formula XVII in which EH is OH or SH by reaction with an appropriate compound of formula IX wherein J is a halogen or OSO₂CF, substituent at the position of ring Ar² at which the bond to oxygen or sulfur is formed. The reaction may be performed by heating in an inert solvent in the presence of a suitable base. The reaction may require, and is preferably performed in, the presence of a catalyst. Suitable inert solvents include ether solvents, for example tetrahydrofuran, 1,4-dioxane, 1,2-dimethoxyethane, or di(2-methoxyethyl)ether, an amide solvent, for example N,N-dimethylformamide, or N-methyl-2-pyrrolidinone, or a basic heterocyclic aromatic solvent, for example pyridine. The preferred solvent is pyridine. Suitable bases include alkali metal alkoxides, or alkali metal carbonates, for example potassium carbonate. Suitable organometallic catalysts include copper or its salts, preferably copper (I) salts, and most preferably copper (I) iodide. The reaction is carried out at a temperature of 0-150° C., and preferably at a temperature of 100-150° C.

Compounds of formula IX, X and XII, XIV, and XVII are either commercially available, known in the literature, or may be prepared by methods known to one skilled in the art. In particular, a compound of formula X in which A is a moiety of formula II: 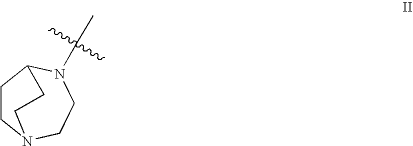
may be prepared by the methods described in: J. Gen. Chem. USSR, 1964, 2222-2228, U.S. Pat. No. 4,895,543, or EP215650.

It will be appreciated by one skilled in the art that certain optional aromatic substituents in the compounds of the invention may be introduced by employing aromatic substitution reactions, or functional group transformations to modify an existing substituent, or a combination thereof. Such reactions may be effected either prior to or immediately following the processes mentioned above, and are included as part of the process aspect of the invention. The reagents and reaction conditions for such procedures are known in the art. Specific examples of procedures which may be employed include, but are not limited to, electrophilic functionalisation of an aromatic ring, for example via nitration, halogenation, or acylation; transformation of a nitro group to an amino group, for example via reduction, such as by catalytic hydrogenation; acylation, alkylation, sulfonylation of an amino or hydroxyl group; replacement of an amino group by another functional group via conversion to an intermediate diazonium salt followed by nucleophilic or free radical substitution of the diazonium salt; or replacement of a halogen by another functional group, for example via nucleophilic or organometallically-catalysed substitution reactions.

Where necessary, hydroxy, amino, or other reactive groups may be protected using a protecting group as described in the standard text “Protecting groups in Organic Synthesis”, 3^{rd} Edition (1999) by Greene and Wuts.

The above described reactions, unless otherwise noted, are usually conducted at a pressure of about one to about three atmospheres, preferably at ambient pressure (about one atmosphere).

Unless otherwise stated, the above described reactions are conducted under an inert atmosphere, preferably under a nitrogen atmosphere.

The compounds of the invention and intermediates may be isolated from their reaction mixtures by standard techniques.

Acid addition salts of the compounds of formula I which may be mentioned include salts of mineral acids, for example the hydrochloride and hydrobromide salts; and salts formed with organic acids such as formate, acetate, Maleate, benzoate, tartrate, and fumarate salts.

Acid addition salts of compounds of formula I may be formed by reacting the free base or a salt, enantiomer or protected derivative thereof, with one or more equivalents of the appropriate acid. The reaction may be carried out in a solvent or medium in which the salt is insoluble or in a solvent in which the salt is soluble, e.g., water, dioxane, ethanol, tetrahydrofuran or diethyl ether, or a mixture of solvents, which may be removed in vacuum or by freeze drying. The reaction may be a metathetical process or it may be carried out on an ion exchange resin.

The compounds of formula I exist in tautomeric or enantiomeric forms, all of which are included within the scope of the invention. The various optical isomers may be isolated by separation of a racemic mixture of the compounds using conventional techniques, e.g. fractional crystallisation, or chiral HPLC. Alternatively the individual enantiomers may be made by reaction of the appropriate optically active starting materials under reaction conditions which will not cause racemisation.

Intermediates

A further aspect of the invention relates to novel intermediates. Intermediates of interest are compounds of formula VI in Scheme 1. These intermediates are useful in the synthesis of compounds of formula I, but their use is not limited to the synthesis of such compounds.

Accordingly, there is also provided a compound of formula VI: 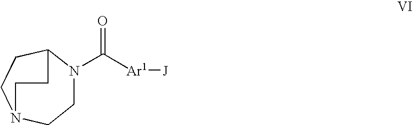
wherein:
Ar¹ is a benzene, furan, or thiophene ring;
J is halogen, or OSO₂CF₃, provided that when Ar¹ is a benzene ring, J may only represent halogen or OSO₂CF₃ in a position meta or para to the carboxamide group; or an enantiomer thereof or pharmaceutically-acceptable salts thereof.

Particular compounds of this aspect of the invention include:
(1,4-diazabicyclo[3.2.2]non-4-yl)(5-bromofuran-2-yl)methanone;
(1,4-diazabicyclo[3.2.2]non-4-yl)(5-bromothiophen-2-yl)methanone;
(1,4-diazabicyclo[3.2.2]non-4-yl)(3-bromophenyl)methanone;
(1,4-diazabicyclo[3.2.2]non-4-yl)(4-bromophenyl)methanone;
(1,4-diazabicyclo[3.2.2]non-4-yl)(3-iodophenyl)methanone;
(1,4-diazabicyclo[3.2.2]non-4-yl)(4-iodophenyl)methanone;
(1,4-diazabicyclo[3.2.2]non-4-yl)(4-bromothiophen-2-yl)methanone;
(1,4-diazabicyclo[3.2.2]non-4-yl)(5-bromothiophen-3-yl)methanone;
(1,4-diazabicyclo[3.2.2]non-4-yl)(5-bromofuran-2-yl)methanone;
(1,4-diazabicyclo[3.2.2]non-4-yl)(5-bromofuran-2-yl)methanone, and
(1,4-diazabicyclo[3.2.2]non-4-yl)(5-bromofuran-2-yl)methanone;
   or enantiomers thereof, or pharmaceutically-acceptable salts thereof.

Intermediate compounds can exist in enantiomeric forms and may be used as purified enantiomers, racemates or mixtures.

### EXAMPLE 1

### (Biphenyl-3-yl)(1,4-diazabicyclo[3.1.2]non-4-yl)methanone

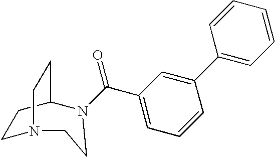

Biphenyl-3-carboxylic acid (52 mg, 0.25 mmol), 1,4-diaza-bicyclo[3.2.2]nonane dihydrochloride (50 mg, 0.25 mmol), 1-hydroxybenzotriazole hydrate (34 mg, 0.25 mmol), O-(benzotriazol-1-yl)-N,N,N′,N′-tetramethyluronium tetrafluoroborate (81 mg, 0.25 mL) and diisopropylethylamine (0.17 mL, 125 mg, 1.0 mmol) in dry N,N-dimethylformamide (2 mL) were stirred at ambient temperature for 20 h. The reaction mixture was poured into 1N sodium hydroxide solution and extracted with ethyl acetate (2×). The ethyl acetate layers were combined and washed with water (2×). The solvent was blown off with a stream of nitrogen to yield (biphenyl-3-yl)(1,4-diazabicyclo[3.2.2]non-4-yl)methanone (62 mg, 77%) as a yellow oil. MS (APCI+) 313 [M+1]+; ¹H-NMR (300 MHz, CDCl₃): δ 7.76-7.61 (4H, m), 7.56-7.33 (5H, m), 4.61-4.53 (1H, m), 3.90-3.73 (1H, m), 3.52-3.43 (1H, m), 3.01-2.78 (6H, m), 2.11-1.59 (4H, m).

### EXAMPLE 2

### (1,4-Diazabicyclo[3.2.2]non-4-yl)(5-phenylfuran-2-yl)methanone

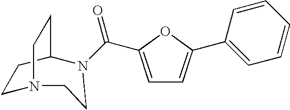

5-Phenylfuran-2-carboxylic acid (49 mg, 0.25 mmol), 1,4-diaza-bicyclo[3.2.2]nonane dihydrochloride (50 mg, 0.25 mmol), 1-hydroxybenzotriazole hydrate (34 mg, 0.25 mmol), O-(benzotriazol-1-yl)-N,N,N′,N′-tetramethyluronium tetrafluoroborate (81 mg, 0.25 mL) and diisopropylethylamine (0.17 mL, 125 mg, 1,0 mmol) in dry N,N-dimethylformamide (2 mL) were stirred at ambient temperature for 20 h. The reaction mixture was poured into 1N sodium hydroxide solution and extracted with ethyl acetate (2×). The ethyl acetate layers were combined and washed with water (2×). The solvent was blown off with a stream of nitrogen to yield (1,4-diazabicyclo[3.2.2]non-4-yl)(5-phenylfuran-2-yl)methanone (26 mg, 34°/O) as a yellow oil. MS (APCI+) 297 [M+1]+. ¹H-NMR (300 MHz, CDCl₃): δ 7.81-7.70 (2H, m), 7.52-7.41 (2H, m), 7.40-7.30 (1H, m), 7.12-7.01 (2H, m), 4.59-4.45 (1H, m), 4.01-3.68 (2H, m), 3.04-2.81 (6H, m), 2.09-1.60 (4H, m).

### EXAMPLE 3

### (1,4-Diazabicyclo[3.2.2]non-4-yl)(5-phenylthiophene-2-yl)methanone

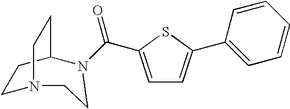

5-Phenylthiophene-2-carboxylic acid (103 mg, 0.50 mmol), 1,4-diaza-bicyclo[3.2.2]nonane dihydrochloride (100 mg, 0.50 mmol), 1-hydroxybenzotriazole hydrate (68 mg, 0.50 mmol), O-(benzotriazol-1-yl)-N,N,N′,N′-tetramethyluronium tetrafluoroborate (161 mg, 0.50 mL) and diisopropylethylamine (0.35 mL, 250 mg, 2.0 mmol) in dry N,N-dimethylformamide (2 mL) were stirred at ambient temperature for 20 h. The reaction mixture was poured into 1N sodium hydroxide solution and extracted with ethyl acetate (2×). The ethyl acetate layers were combined and washed with water (2×). The solvent was blown off with a stream of nitrogen to yield (1,4-diazabicyclo[3.2.2]non-4-yl)(5-phenylthiophene-2-yl)methanone (122 mg, 78%) as a tan oil. MS (APCI+) 313 [M+1]+. ¹H-NM (300 MHz, CDCl₃): δ 7.74-7.66 (2H, m), 7.53-7.32 (5H, m), 4.53-4.39 (1H, m), 3.89-3.72 (2H, m), 3.01-2.83 (6H, m), 2.06-1.85 (2H, m), 1.82-1.64 (2H, m).

### EXAMPLE 4

### (1,4-Diaza-bicyclo[3.2.2]non-4-yl)-(5-pyridin-2-yl-thiophen-2-yl)-methanone

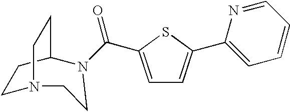

5-(2-Pyridyl)thiophene-2-carboxylic acid (42 mg, 0.25 mmol), 1,4-diaza-bicyclo[3.2.2]nonane dihydrochloride (50 mg, 0.25 mmol), 1-hydroxybenzotriazole hydrate (34 mg, 0.25 mmol), O-(benzotriazol-1-yl)-N,N,N′,N′-tetramethyluronium tetrafluoroborate. (81 mg, 0.25 mmol) and diisopropylethylamine (0.17 mL, 129 mg, 1.0 mmol) in dry N,N-dimethylformamide (1.5 mL) were stirred at ambient temperature for 24 h. The reaction mixture was poured into 1N sodium hydroxide solution and extracted with ethyl acetate. The ethyl acetate layer was washed with 1N NaOH (1×), water (4×), brine (1×), and dried over Na₂SO₄. After filtration, the solvent was removed in vacuo to yield 41 mg of product. The reaction mixture was chromatographed with 100% EtOAc to 90:10 EtOAc:7N NH₃/MeOH to give (1,4-diaza-bicyclo[3.2.2]non-4-yl)-(5-pyridin-2-yl-thiophen-2-yl)-methanone (40 mg, 51%) as a colorless oil. MS (APCI+) 314 [M+1]+; ¹H-NMR (300 MHz, CDCl₃): δ 8.58 (1H, d), 7.77-7.65 (2H, m), 7.50 (1H, d), 7.33 (1H, d), 7.21-7.17 (1H, m), 4.68 (1H, s), 3.90 (2H, t), 3.16-2.98 (6H, m), 2.10-2.04 (2H, m), 1.91 (1H, s), 1.86-1.75 (2H, m).

### EXAMPLE 5

### (1,4-Diaza-bicyclo[3.2.2]non-4-yl](5-pyridin-3-yl-thiophen-2-yl)-methanone

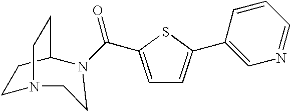

5-Bromothiophene-2-carboxylic acid (104 mg, 0.502 mmol), 1,4-diaza-bicyclo[3.2.2]nonane dihydrochloride (100 mg, 0.502 mmol), 1-hydroxybenzotriazole hydrate (68 mg, 0.502 mmol), O-(benzotriazol-1-yl)-N,N,N′,N′-tetramethyluronium tetrafluoroborate (161 mg, 0.502 mmol) and diisopropylethylamine (0.350 mL, 260 mg, 2.01 mmol) in dry N,N-dimethylformamide (3.0 mL) were stirred at ambient temperature for 24 h. The reaction mixture was poured into 1N sodium hydroxide solution and extracted with ethyl acetate. The ethyl acetate layer was washed with 1N NaOH (1×), water (4×), brine (1×), and dried over Na₂SO₄. After filtration, the solvent was removed in vacuo to yield (5-Bromo-thiophen-2-yl)-(1,4-diaza-bicyclo[3.2.2]non-4-yl)-methanone (123 mg, 78%). The product was taken directly into the next reaction without any purification.

A conical microwave vessel was charged with (5-bromo-thiophen-2-yl)-(1,4-diaza-bicyclo[3.2.2]non-4-yl)-methanone (123 mg, 0.390 mmol), 3-pyridylboronic acid (58 mg, 0.468 mmol), dichlorobis(triphenylphosphine)-palladium (II) (2.7 mg, 0.0039 mmol), cesium carbonate (152 mg, 0.468 mmol) and 7:3:2 DME/H₂O/EtOH (2.5 mL). The reaction was run in the Smith Synthesizer at 160° C. for 150 seconds. The reaction mixture was filtered through a pad of diatomaceous earth and washed with EtOAc (3×). The combined ethyl acetate layers were washed with H₂O, dried over Na₂SO₄, filtered and concentrated to give 62 mg of product. The mixture was chromatographed with 100% EtOAc to 90:10 EtOAc: 7N NH₃/MeOH to give (1,4-diaza-bicyclo[3.2.2]non-4-yl)-(5-pyridin-3-yl-thiophen-2-yl)-methanone (28 mg, 23%) as a white solid. MS (APCI+) 314 [M+1]+; ¹H-NMR (300 MHz, CDCl₃): δ 8.81 (1H, d), 8.49 (1H, dd), 8.05-7.78 (1H, m), 7.28-7.21 (3H, m), 4.58 (1H, s), 3.85-3.76 (2H, m), 3.09-2.91 (6H, m), 2.03-2.01 (2H, m), 1.80-1.69 (2H, m).

### EXAMPLE 6

### (1,4-Diaza-bicyclo[3.2.2]non-4-yl)-(3-thiophen-2-yl-phenyl)-methanone

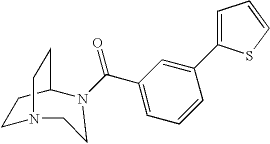

3-Bromobenzoic acid (101 mg, 0.502 mmol), 1,4-diaza-bicyclo[3.2.2]nonane dihydrochloride (100 mg, 0.502 mmol), 1-hydroxybenzotriazole hydrate (68 mg, 0.502 mmol), O-(benzotriazol-1-yl)-N,N,N′,N′-tetramethyluronium tetrafluoroborate (161 mg, 0.502 mmol) and diisopropylethylamine (0.350 mL, 260 mg, 2.01 mmol) in dry N,N-dimethylformamide (3.0 mL) were stirred at ambient temperature for 24 h. The reaction mixture was poured into 1N sodium hydroxide solution and extracted with ethyl acetate. The ethyl acetate layer was washed with 1N NaOH (1×), water (4×), brine (1×), and dried over Na₂SO₄. After filtration, the solvent was removed in vacuo to yield (3-bromo-phenyl)-(1,4-diaza-bicyclo[3.2.2]non-4-yl)-methanone (108 mg, 70%). The product was taken directly into the next reaction without any purification.

A conical microwave vessel was charged with (3-Bromo-phenyl)-(1,4-diaza-bicyclo[3.2.2]non-4-yl)-methanone (108 mg, 0.349 mmol), 2-thiopheneboronic acid (54 mg, 0.419 mmol), dichlorobis(triphenylphosphine)-palladium (II) (2.4 mg, 0.00349 mmol), cesium carbonate (137 mg, 0.419 mmol) and 7:3:2 DME/H₂O/EtOH (2.5 mL). The reaction was run in the Smith Synthesizer at 160° C. for 150 seconds. The reaction mixture was filtered through a pad of diatomaceous earth and washed with EtOAc (3×). The combined ethyl acetate layers were washed with H₂O, dried over Na₂SO₄, filtered and concentrated to give 98 mg of product. The mixture prepared on the Gilson reverse phase HPLC to give (1,4-diaza-bicyclo[3.2.2]non-4-yl)-(3-thiophen-2-yl-phenyl)-methanone (90 mg, 83%) as a colorless oil, TFA salt. MS (APCI+) 313 [M+1]+; ¹H-NMR (300 MHz, CDCl₃): δ 12.04 (1H, s), 7.72 (1H, d), 7.62 (1H, s), 7.47 (1H, t), 7.35-7.29 (3H, m), 7.11 (1H, t), 5.05 (1H, s), 3.93 (2H, s), 3.55-3.52 (6H, m), 2.39 (2H, s), 2.20 (2H, s).

### EXAMPLE 7

### (1,4-Diaza-bicyclo[3.2.2]non-4-yl)-(5-thiophen-2-yl-furan-2-yl)-methanone

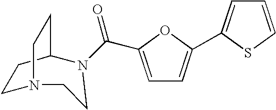

5-Bromo-2-furoic acid (96 mg, 0.502 mmol), 1,4-diaza-bicyclo[3.2.2]nonane dihydrochloride (100 mg, 0.502 mmol), 1-hydroxybenzotriazole hydrate (68 mg, 0.502 mmol), O-(benzotriazol-1-yl)-N,N,N′,N′-tetramethyluronium tetrafluoroborate (161 mg, 0.502 mmol) and diisopropylethylamine (0.350 mL, 260 mg, 2.01 mmol) in dry N,N-dimethylformamide (3.0 mL) were stirred at ambient temperature for 24 h. The reaction mixture was poured into 1N sodium hydroxide solution and extracted with ethyl acetate. The ethyl acetate layer was washed with 1N NaOH (1×), water (4×), brine (1×), and dried over Na₂SO₄. After filtration, the solvent was removed in vacuo to yield (5-bromo-furan-2-yl)-(1,4-diaza-bicyclo[3.2.2]non-4-yl)-methanone (84 mg, 56%). The product was taken directly into the next reaction without any purification.

A conical microwave vessel was charged with (5-bromo-furan-2-yl)-(1,4-diaza-bicyclo[3.2.2]non-4-yl)-methanone (84 mg, 0.281 mmol), 2-thiopheneboronic acid (43 mg, 0.337 mmol), dichlorobis(triphenylphosphine)-palladium (II) (2.0 mg, 0.00281 mmol), cesium carbonate (110 mg, 0.337 mmol) and 7:3:2 DME/H₂O/EtOH (2.5 mL). The reaction was run in the Smith Synthesizer at 160° C. for 150 seconds. The reaction mixture was filtered through a pad of diatomaceous earth and washed with EtOAc (3×). The combined ethyl acetate layers were washed with H₂O, dried over Na₂SO₄, filtered and concentrated to give 70 mg of product. The mixture prepared on the Gilson reverse phase HPLC to give (1,4-diaza-bicyclo[3.2.2]non-4-yl)-(5-thiophen-2-yl-furan-2-yl)-methanone (33 mg, 39%) as a colorless oil, TFA salt. MS (APCI+) 303 [M+1]+; ¹H-NMR (300 MHz, CDCl₃): δ 12.55 (1H, s), 7.35 (2H, t), 7.11 (1H, dd), 6.50 (1H, d), 6.54 (1H, s), 5.03-5.01 (1H, m), 4.27 (2H, s), 3.69-3.47 (6H, m), 2.46 (2H, s), 2.29-2.26 (2H, m).

### EXAMPLE 8

### [5-(4-Chlorophenyl)furan-2-yl](1,4-diaza-bicyclo[3.2.2]non-4-yl)methanone

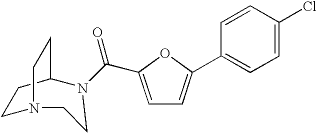

5-(4-Chlorophenyl)furan-2-carboxylic acid (56 mg, 0.25 mmol), 1,4-diaza-bicyclo[3.2.2]nonane dihydrochloride (50 mg, 0.25 mmol), 1-hydroxybenzotriazole hydrate (34 mg, 0.25 mmol), O-(benzotriazol-1-yl)-N,N,N′,N′-tetramethyl uronium tetrafluoroborate (81 mg, 0.25 mL) and diisopropylethylamine (0.17 mL, 125 mg, 1.0 mmol) in dry N,N-dimethylformamide (2 mL) were stirred at ambient temperature for 42 h. The reaction mixture was poured into 1N sodium hydroxide solution and extracted with ethyl acetate. The ethyl acetate layer was washed with 1N NaOH (1×), water (4×), brine (1×) and dried over Na₂SO₄. The solvent was removed in vacuo to yield [5-(4-chlorophenyl)furan-2-yl](1,4-diaza-bicyclo[3.2.2]non-4-yl)methanone (76 mg, 92%) as a beige semisolid. MS (APCI+) 331/333 [M+1]+. ¹H-NMR (300 MHz, CDCl₃): δ 7.83-7.74 (2H, d), 7.58-7.49 (2H, d), 7.18-7.11 (1H, m), 7.11-7.04 (1H, m), 4.55-4.46 (1H, m), 3.97-3.68 (2H, m), 3.04-2.84 (6H, m), 2.09-1.89 (2H, m), 1.89-1.61 (2H, m).

Pharmaceutical Compositions

A further aspect of the invention relates to a pharmaceutical composition for treating or preventing a condition or disorder as exemplified below arising from dysfunction of nicotinic acetylcholine receptor neurotransmission in a mammal, preferably a human, comprising an amount of a compound of formula I, an enantiomer thereof or a pharmaceutically-acceptable salt thereof, effective in treating or preventing such disorder or condition in admixture with an inert pharmaceutically-acceptable diluent or carrier.

For the above-mentioned uses the dosage administered will, of course, vary with the compound employed, the mode of administration and the treatment desired. However, in general, satisfactory results are obtained when the compounds of the invention are administered at a daily dosage of from about 0.1 mg to about 20 mg per kg of animal body weight, preferably given in divided doses 1 to 4 times a day or in sustained release form. For man, the total daily dose is in the range of from 5 mg to 1,400 mg, more preferably from 10 mg to 100 mg, and unit dosage forms suitable for oral administration comprise from 2 mg to 1,400 mg of the compound admixed with a solid or liquid pharmaceutical carrier or diluent.

The compounds of formula I, or an enantiomer thereof, or pharmaceutically-acceptable salts thereof, may be used on their own or in the form of appropriate medicinal preparations for enteral or parenteral administration. According to a further aspect of the invention, there is provided a pharmaceutical composition including preferably less than 80% and more preferably less than 50% by weight of a compound of the invention in admixture with an inert pharmaceutically-acceptable diluent or carrier.

Examples of diluents and carriers are:
for tablets and dragees: lactose, starch, talc, stearic acid;
for capsules: tartaric acid or lactose;
for injectable solutions: water, alcohols, glycerin, vegetable oils;
for suppositories: natural or hardened oils or waxes.

There is also provided a process for the preparation of such a pharmaceutical composition which comprises mixing the ingredients.

A further aspect of the invention is the use of a compound according to the invention, an enantiomer thereof or a pharmaceutically-acceptable salt thereof, in the manufacture of a medicament for the treatment or prophylaxis of one of the diseases or conditions mentioned herein; and a method of treatment or prophylaxis of one of the above mentioned diseases or conditions, which comprises administering a therapeutically effective amount of a compound according to the invention, or an enantiomer thereof or a pharmaceutically-acceptable salt thereof, to a patient.

Compounds according to the invention are agonists of nicotinic acetylcholine receptors. While not being limited by theory, it is believed that agonists of the α₇ nAChR (nicotinic acetylcholine receptor) subtype should be useful in the treatment or prophylaxis of psychotic disorders and intellectual impairment disorders, and have advantages over compounds which are or are also agonists of the α₄ nAChR subtype. Therefore, compounds which are selective for the α₇ nAChR subtype are preferred. The compounds of the invention are indicated as pharmaceuticals, in particular in the treatment or prophylaxis of psychotic disorders and intellectual impairment disorders. Examples of psychotic disorders include schizophrenia, mania and manic depression, and anxiety. Examples of intellectual impairment disorders include Alzheimer's disease, learning deficit, cognition deficit, attention deficit, Lewy Body Dementia, memory loss, and Attention Deficit Hyperactivity Disorder. The compounds of the invention may also be useful as analgesics in the treatment of pain (including chronic pain) and in the treatment or prophylaxis of Parkinson's disease, Huntington's disease, Tourette's syndrome, and neurodegenerative disorders in which there is loss of cholinergic synapses. The compounds may further be indicated for the treatment or prophylaxis of jetlag, for use in inducing the cessation of smoking, and for the treatment or prophylaxis of nicotine addiction (including that resulting from exposure to products containing nicotine).

It is also believed that compounds according to the invention are useful in the treatment and prophylaxis of ulcerative colitis and irritable bowel disease.

Pharmacology

The pharmacological activity of the compounds of the invention may be measured in the tests set out below:

Test A—Assay for Affinity at α₇ nAChR Subtype

¹²⁵I-α-Bungarotoxin (BTX) Binding to Rat Hippocampal Membranes.

Rat hippocampi were homogenized in 20 volumes of cold homogenisation buffer (HB: concentrations of constituents (mM): tris(hydroxymethyl)aminomethane 50; MgCl₂ 1; NaCl 120; KCl 5: pH 7.4). The homogenate was centrifuged for 5 minutes at 1000×g, the supernatant was saved and the pellet re-extracted. The pooled supernatants were centrifuged for 20 minutes at 12000×g, washed, and re-suspended in HB. Membranes (30-80 μg) were incubated with 5 nM [¹²⁵I]α-BTX, 1 mg/mL BSA (bovine serum albumin), test drug, and either 2 mM CaCl₂ or 0.5 mM EGTA [ethylene glycol-bis(β-aminoethylether)] for 2 hours at 21° C., and then filtered and washed 4 times over Whatman glass fibre filters (thickness C) using a Brandel cell harvester. Pre-treating the filters for 3 hours with 1% (BSA/0.01% PEI (polyethyleneimine) in water was critical for low filter blanks (0.07% of total counts per minute). Non-specific binding was described by 100 μM (−)-nicotine, and specific binding was typically 75%.

Test B—Assay for Affinity to the a 4 nAChR Subtype

[³H]-(−)-Nicotine Binding.

Using a procedure modified from Martino-Barrows and Kellar (Mol Pharm (1987) 31:169-174), rat brain (cortex and hippocampus) was homogenised as in the [¹²⁵I]α-BTX binding assay, centrifuged for 20 minutes at 12,000×g, washed twice, and then re-suspended in HB containing 100 μM diisopropyl fluorophosphate. After 20 minutes at 4° C., membranes (approximately 0.5 mg) were incubated with 3 nM [³H]-(−)-nicotine, test drug, 1 μM atropine, and either 2 mM CaCl₂ or 0.5 mM EGTA for 1 hour at 4° C., and then filtered over Whatman glass fibre filters (thickness C) (pre-treated for 1 hour with 0.5% PEI) using a Brandel cell harvester. Non-specific binding was described by 100 μM carbachol, and specific binding was typically 84%.

Binding Data Analysis for Tests A and B

IC₅₀ values and pseudo Hill coefficients (n_{H}) were calculated using the non-linear curve fitting program ALLFIT (DeLean A, Munson P J and Rodbard D (1977) Am. J. Physiol., 235:E97-E102). Saturation curves were fitted to a one site model, using the non-linear regression program ENZFITTER (Leatherbarrow, R. J. (1987)), yielding K_{D} values of 1.67 and 1.70 nM for the ¹²⁵I-α-BTX and [³H]-(−)-nicotine ligands respectively. Kᵢ values were estimated using the general Cheng-Prusoff equation:
Kᵢ=[IC₅₀/((2+([ligand]/K_{D}])ⁿ)^{1/n}−1)
where a value of n=1 was used whenever n_{H}<1.5 and a value of n=2 was used when n_{H}≧1.5. Samples were assayed in triplicate and were typically ±5%. Kᵢ values were determined using 6 or more drug concentrations. The compounds of the invention are compounds with binding affinities (Kᵢ) of less than 10 μM in either Test A or Test B, indicating that they are expected to have useful therapeutic activity.

The compounds of the invention have the advantage that they may be less toxic, be more efficacious, be longer acting, have a broader range of activity, be more potent, produce fewer side effects, are more easily absorbed or have other useful pharmacological properties.

1. A compound of formula I: 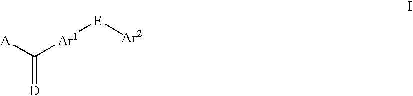
wherein:
A is a moiety of formula II: 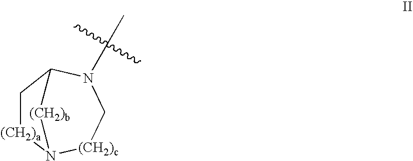
D is oxygen or sulfur;
E is a single bond, oxygen, sulfur, or NR³;
Ar¹ is a 5- or 6-membered aromatic heterocyclic ring having 1, 2 or 3 heteroatoms selected from nitrogen, oxygen or sulfur where not more than one of said heteroatoms is oxygen or sulfur, or
Ar¹ is phenyl;
Ar² is a 5- or 6-membered aromatic heterocyclic ring having 1, 2 or 3 heteroatoms selected from nitrogen, oxygen or sulfur where not more than one of said heteroatoms is oxygen or sulfur, or
Ar² is phenyl, or
Ar² is an 8- or 9-, or 10-membered fused aromatic carbocyclic ring or fused aromatic heterocyclic ring having 1, 2 or 3 heteroatoms selected from nitrogen, oxygen or sulfur where not more than one of said heteroatoms is oxygen or sulfur, or an 8- or 9-, or 10-membered aromatic carbocyclic ring;
the rings Ar¹ and Ar² are substituted with 0, 1, 2 or 3 substituents selected from: halogen, C₁₋₄alkyl, C₂₋₄alkenyl, C₂₋₄alkynyl, CN, NO₂, CF₃NR¹R², CH₂NR¹R², OR², CH₂OR² or CO₂R³;
R¹ and R² at each occurrence are independently selected from hydrogen, C₁₋₄alkyl, aryl, heteroaryl, C(O)R³, C(O)NHR³, CO₂R³ or SO₂R³, or
R¹ and R² in combination is —(CH₂)ⱼG(CH₂)ₖ— wherein G is oxygen, sulfur, NR³, or a bond;
a, b and c are each 1 or 2;
j is 2, 3 or 4;
k is 0, 1 or 2, and
R³ at each occurrence is independently selected from hydrogen, C₁₋₄alkyl, aryl, or heteroaryl;
or a diastereoisomer, enantiomer or pharmaceutically-acceptable salt thereof.

2. A compound according to claim 1, wherein D is oxygen.

3. A compound according to claim 2, wherein E is a single bond.

4. A compound according to claim 2, wherein E is oxygen or NR³.

5. A compound according to claim 1, wherein A is 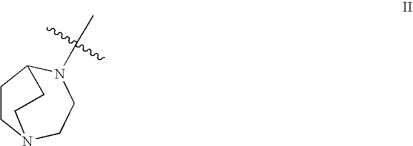
or a diastereoisomer, enantiomer or pharmaceutically-acceptable salt thereof.

6. A compound of claim 1, wherein
Ar¹ is a 5- or 6-membered aromatic heterocyclic ring having 1 or 2 heteroatoms selected from nitrogen, oxygen or sulfur where not more than one of said heteroatoms is oxygen or sulfur, or
Ar¹ is phenyl,
or a diastereoisomer, enantiomer or pharmaceutically-acceptable salt thereof.

7. A compound according to claim 6 wherein Ar¹ is a benzene ring, furan ring or thiophene ring.

8. A compound according to claim 1, wherein
Ar² is a 5- or 6-membered aromatic heterocyclic ring having 1 or 2 heteroatoms selected from nitrogen, oxygen or sulfur where not more than one of said heteroatoms is oxygen or sulfur, or a phenyl,
or a diastereoisomer, enantiomer or pharmaceutically-acceptable salt thereof.

9. A compound according to claim 8, wherein Ar² is a benzene ring, furan ring, thiophene ring, or pyridine ring.

10. A compound according to claim 1, wherein
the -EAr² and the C(=D)A moieties on Ar¹ are positioned in a 1,3-relationship relative to each other;
or a diastereoisomer, enantiomer or pharmaceutically-acceptable salt thereof.

11. A compound according to claim 1, wherein Ar¹ or Ar² is substituted with 0 or 1 substituents selected from: halogen, C₁₋₄alkyl, C₂₋₄alkenyl, C₂₋₄alkynyl, CN, NO₂, NR¹R², CH₂NR¹R², OR³, CH₂OR³, CO₂R³ or CF₃;
or a diastereoisomer, enantiomer or pharmaceutically-acceptable salt thereof.

12. A compound according to claim 1, wherein A is a moiety of formula II: 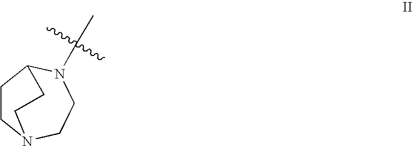
D is oxygen;
E is a single bond;
Ar¹ is a 5- or 6-membered aromatic heterocyclic ring having 1, 2 or 3 heteroatoms selected from nitrogen, oxygen or sulfur where not more than 1 of said heteroatoms is oxygen or sulfur, or
Ar¹ is phenyl
Ar² is a 5- or 6-membered aromatic heterocyclic ring having 1, 2 or 3 heteroatoms selected from nitrogen, oxygen or sulfur where not more than 1 of said heteroatoms is oxygen or sulfur, or
Ar² is phenyl,
or a diastereoisomer, enantiomer or pharmaceutically-acceptable salt thereof.

13. A compound of claim 12, wherein Ar¹ is a benzene ring, furan ring or thiophene ring.

14. A compound according to claim 1, having the groups -EAr² and —C(═O)A, positioned in a 1,3-relationship relative to each other and wherein Ar² has 0 or 1 substituents selected from: halogen, C₁₋₄alkyl, C₂₋₄alkenyl, C₂₋₄alkynyl, CN, NO₂, NR¹R², CH₂NR¹R², OR¹, CH₂OR¹, CO₂R or CF₃;
or a diastereoisomer, enantiomer or pharmaceutically-acceptable salt thereof.

15. A compound according to claim 1, selected from:
(1,4-diazabicyclo[3.2.2]non-4-yl)(biphenyl-3-yl)methanone;
(1,4-diazabicyclo[3.2.2]non-4-yl)(3-(2-pyridyl)phenyl)methanone;
(1,4-diazabicyclo[3.2.2]non-4-yl)(3-(3-pyridyl)phenyl)methanone;
(1,4-diazabicyclo[3.2.2]non-4-yl)(3-(4-pyridyl)phenyl)methanone;
(1,4-diazabicyclo[3.2.2]non-4-yl)(3-(furan-2-yl)phenyl)methanone;
(1,4-diazabicyclo[3.2.2]non-4-yl)(3-(furan-3-yl)phenyl)methanone;
(1,4-diazabicyclo[3.2.2]non-4-yl)(3-(thiophen-2-yl)phenyl)methanone;
(1,4-diazabicyclo[3.2.2]non-4-yl)(3-(thiophen-3-yl)phenyl)methanone;
(1,4-diazabicyclo[3.2.2]non-4-yl)(biphenyl-4-yl)methanone;
(1,4-diazabicyclo[3.2.2]non-4-yl)(4-(2-pyridyl)phenyl)methanone;
(1,4-diazabicyclo[3.2.2]non-4-yl)(4-(3-pyridyl)phenyl)methanone;
(1,4-diazabicyclo[3.2.2]non-4-yl)(4-(4-pyridyl)phenyl)methanone;
(1,4-diazabicyclo[3.2.2]non-4-yl)(4-(furan-2-yl)phenyl)methanone;
(1,4-diazabicyclo[3.2.2]non-4-yl)(4-(furan-3-yl)phenyl)methanone;
(1,4-diazabicyclo[3.2.2]non-4-yl)(4-(thiophen-2-yl)phenyl)methanone;
(1,4-diazabicyclo[3.2.2]non-4-yl)(4-(thiophen-3-yl)phenyl)methanone;
(1,4-diazabicyclo[3.2.2]non-4-yl)(5-phenylfuran-2-yl)methanone;
(1,4-diazabicyclo[3.2.2]non-4-yl)(5-(2-pyridyl)furan-2-yl)methanone;
(1,4-diazabicyclo[3.2.2]non-4-yl)(5-(3-pyridyl)furan-2-yl)methanone;
(1,4-diazabicyclo[3.2.2]non-4-yl)(5-(4-pyridyl)furan-2-yl)methanone;
(1,4-diazabicyclo[3.2.2]non-4-yl)(5-(furan-2-yl)furan-2-yl)methanone;
(1,4-diazabicyclo[3.2.2]non-4-yl)(5-(furan-3-yl)furan-2-yl)methanone;
(1,4-diazabicyclo[3.2.2]non-4-yl)(5-(thiophen-2-yl)furan-2-yl)methanone;
(1,4-diazabicyclo[3.2.2]non-4-yl)(5-(thiophen-3-yl)furan-2-yl)methanone;
(1,4-diazabicyclo[3.2.2]non-4-yl)(2-phenylthiophen-4-yl)methanone;
(1,4-diazabicyclo[3.2.2]non-4-yl)(2-(2-pyridyl)thiophen-4-yl)methanone;
(1,4-diazabicyclo[3.2.2]non-4-yl)(2-(3-pyridyl)thiophen-4-yl)methanone;
(1,4-diazabicyclo[3.2.2]non-4-yl)(2-(4-pyridyl)thiophen-4-yl)methanone;
(1,4-diazabicyclo[3.2.2]non-4-yl)(2-(furan-2-yl)thiophen-4-yl)methanone;
(1,4-diazabicyclo[3.2.2]non-4-yl)(2-(furan-3-yl)thiophen-4-yl)methanone;
(1,4-diazabicyclo[3.2.2]non-4-yl)(2-(thiophen-2-yl)thiophen-4-yl)methanone;
(1,4-diazabicyclo[3.2.2]non-4-yl)(2-(thiophen-3-yl)thiophen-4-yl)methanone;
(1,4-diazabicyclo[3.2.2]non-4-yl)(4-phenylfuran-2-yl)methanone;
(1,4-diazabicyclo[3.2.2]non-4-yl)(4-(2-pyridyl)furan-2-yl)methanone;
(1,4-diazabicyclo[3.2.2]non-4-yl)(4-(3-pyridyl)furan-2-yl)methanone;
(1,4-diazabicyclo[3.2.2]non-4-yl)(4-(4-pyridyl)furan-2-yl)methanone;
(1,4-diazabicyclo[3.2.2]non-4-yl)(4-(furan-2-yl)furan-2-yl)methanone;
(1,4-diazabicyclo[3.2.2]non-4-yl)(4-(furan-3-yl)furan-2-yl)methanone;
(1,4-diazabicyclo[3.2.2]non-4-yl)(4-(thiophen-2-yl)furan-2-yl)methanone;
(1,4-diazabicyclo[3.2.2]non-4-yl)(4-(thiophen-3-yl)furan-2-yl)methanone;
(1,4-diazabicyclo[3.2.2]non-4-yl)(5-phenylthiophen-2-yl)methanone;
(1,4-diazabicyclo[3.2.2]non-4-yl)(5-(2-pyridyl)thiophen-2-yl)methanone;
(1,4-diazabicyclo[3.2.2]non-4-yl)(5-(3-pyridyl)thiophen-2-yl)methanone;
(1,4-diazabicyclo[3.2.2]non-4-yl)(5-(4-pyridyl)thiophen-2-yl)methanone;
(1,4-diazabicyclo[3.2.2]non-4-yl)(5-(furan-2-yl)thiophen-2-yl)methanone;
(1,4-diazabicyclo[3.2.2]non-4-yl)(5-(furan-3-yl)thiophen-2-yl)methanone;
(1,4-diazabicyclo[3.2.2]non-4-yl)(5-(thiophen-2-yl)thiophen-2-yl)methanone;
(1,4-diazabicyclo[3.2.2]non-4-yl)(5-(thiophen-3-yl)thiophen-2-yl)methanone;
(1,4-diazabicyclo[3.2.2]non-4-yl)(2-phenylfuran-4-yl)methanone;
(1,4-diazabicyclo[3.2.2]non-4-yl)(2-(2-pyridyl)furan-4-yl)methanone;
(1,4-diazabicyclo[3.2.2]non-4-yl)(2-(3-pyridyl)furan-4-yl)methanone;
(1,4-diazabicyclo[3.2.2]non-4-yl)(2-(4-pyridyl)furan-4-yl)methanone;
(1,4-diazabicyclo[3.2.2]non-4-yl)(2-(furan-2-yl)furan-4-yl)methanone;
(1,4-diazabicyclo[3.2.2]non-4-yl)(2-(furan-3-yl)furan-4-yl)methanone;
(1,4-diazabicyclo[3.2.2]non-4-yl)(2-(thiophen-2-yl)furan-4-yl)methanone;
(1,4-diazabicyclo[3.2.2]non-4-yl)(2-(thiophen-3-yl)furan-4-yl)methanone;
(1,4-diazabicyclo[3.2.2]non-4-yl)(4-phenylthiophen-2-yl)methanone;
(1,4-diazabicyclo[3.2.2]non-4-yl)(4-(2-pyridyl)thiophen-2-yl)methanone;
(1,4-diazabicyclo[3.2.2]non-4-yl)(4-(3-pyridyl)thiophen-2-yl)methanone;
(1,4-diazabicyclo[3.2.2]non-4-yl)(4-(4-pyridyl)thiophen-2-yl)methanone;
(1,4-diazabicyclo[3.2.2]non-4-yl)(4-(furan-2-yl)thiophen-2-yl)methanone;
(1,4-diazabicyclo[3.2.2]non-4-yl)(4-(furan-3-yl)thiophen-2-yl)methanone;
(1,4-diazabicyclo[3.2.2]non-4-yl)(4-(thiophen-2-yl)thiophen-2-yl)methanone, or
(1,4-diazabicyclo[3.2.2]non-4-yl)(4-(thiophen-3-yl)thiophen-2-yl)methanone,
or a diastereoisomer, enantiomer or pharmaceutically-acceptable salt thereof.

16-18. (canceled)

19. A method of treatment or prophylaxis of psychotic disorders, intellectual impairment disorders, human diseases or conditions in which activation of the α7 nicotinic receptor is beneficial, Alzheimer's disease, learning deficit, cognition deficit, attention deficit, memory loss, Lewy Body Dementia, Attention Deficit Hyperactivity Disorder, anxiety, schizophrenia, mania or manic depression, Parkinson's disease, Huntington's disease, Tourette's syndrome, neurodegenerative disorders in which there is loss of cholinergic synapse, jetlag, cessation of smoking, nicotine addiction including that resulting from exposure to products containing nicotine, pain, or ulcerative colitis which method comprises administering a therapeutically effective amount of a compound as defined in claim 1.

20. A pharmaceutical composition comprising a compound of formula I, as defined in claim 1, together with at least one pharmaceutically-acceptable excipient or diluent.

21. A process for the preparation of a compound of formula I, as defined in claim 1, which comprises:
reacting a compound of formula VI: 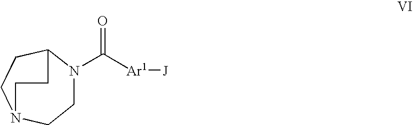
wherein J represents halogen, or OSO₂CF₃ substituent at the position of ring Ar¹ at which the bond to ring Ar² is formed with a organometallic compound of formula VII;
Ar²-M VII
in the presence of a organometallic catalyst and solvent.

22. A compound of formula VI: 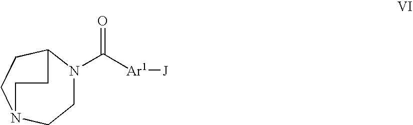
wherein:
Ar¹ is a benzene, furan, or thiophene ring;
J is halogen, or OSO₂CF₃, provided that when Ar¹ is a benzene ring, J may only represent halogen or OSO₂CF₃ in a position meta or para to the carboxamide group; or an enantiomer thereof or pharmaceutically-acceptable salts thereof.

23. A compound according to claim 22, selected from:
(1,4-diazabicyclo[3.2.2]non-4-yl)(5-bromofuran-2-yl)methanone;
(1,4-diazabicyclo[3.2.2]non-4-yl)(5-bromothiophen-2-yl)methanone;
(1,4-diazabicyclo[3.2.2]non-4-yl)(3-bromophenyl)methanone;
(1,4-diazabicyclo[3.2.2]non-4-yl)(4-bromophenyl)methanone;
(1,4-diazabicyclo[3.2.2]non-4-yl)(3-iodophenyl)methanone;
(1,4-diazabicyclo[3.2.2]non-4-yl)(4-iodophenyl)methanone;
(1,4-diazabicyclo[3.2.2]non-4-yl)(4-bromothiophen-2-yl)methanone;
(1,4-diazabicyclo[3.2.2]non-4-yl)(5-bromothiophen-3-yl)methanone;
(1,4-diazabicyclo[3.2.2]non-4-yl)(5-bromofuran-2-yl)methanone;
(1,4-diazabicyclo[3.2.2]non-4-yl)(5-bromofuran-2-yl)methanone, and
(1,4-diazabicyclo[3.2.2]non-4-yl)(5-bromofuran-2-yl)methanone;
or enantiomers thereof, or pharmaceutically-acceptable salts thereof.
